(19) United States
(12) Patent Application Publication
(10) Pub. No.: US 2008/0293051 A1
(43) Pub. Date: Nov. 27, 2008

(54) PROXIMITY LIGATION ASSAY

(75) Inventors: Matthew Levy, Austin, TX (US); Andrew D. Ellington, Austin, TX (US); Supriya Pai, Austin, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 11/834,546

(22) Filed: Aug. 6, 2007

Related U.S. Application Data
(63) Continuation-in-part of application No. 11/512,439, filed on Aug. 30, 2006.
(60) Provisional application No. 60/712,600, filed on Aug. 30, 2005.

(51) Int. Cl. C12Q 1/68

(52) U.S. Cl. 435/6

(57) ABSTRACT

The present invention relates to compositions and methods for sensitive, rapid and convenient assays to detect and/or quantify one or more target using ribonucleic acid as probes, wherein the method includes binding a first and a second ribonucleic acid probe, each of which binds specifically to the target, wherein the first and second probes each comprise a ribonucleic acid tail; ligating the first and second ribonucleic acids tails thereby producing a ligated ribonucleic acid template; and performing amplification of the ribonucleic acid template across the first and second ribonucleic acids.

### CROSS-REFERENCE TO RELATED APPLICATIONS

This application is a Continuation-in-Part of, and claims priority to, U.S. patent application Ser. No. 11/512,439, filed Aug. 30, 2006, which claims priority to U.S. Provisional Patent Application Ser. No. 60/712,600, filed Aug. 30, 2005, the entire contents of each of which are incorporated herein by reference.

### TECHNICAL FIELD OF THE INVENTION

The present invention relates in general to the field of molecular biology, particularly, methods for detecting a target in a sample.

### STATEMENT OF FEDERALLY FUNDED RESEARCH

This invention was made with U.S. Government support. The government has certain rights in this invention.

### INCORPORATION-BY-REFERENCE OF MATERIALS FILED ON COMPACT DISC

None.

### BACKGROUND OF THE INVENTION

Without limiting the scope of the invention, its background is described in connection with proximity ligation assay.

Proximity probing, also termed proximity ligation, is a technique capable of detecting proximity probes and is used for specific, sensitive and rapid detection of macromolecules such as proteins. Proximity ligation relies on two adherent molecules (antibodies, peptides, proteins, aptamers) bound to individual non-overlapping synthetic oligonucleotides to be brought into spatial proximity through binding an analyte. A third oligonucleotide is introduced that acts as a bridge to bring the two non-overlapping oligonucleotides together allowing a DNA ligase to complete a contiguous DNA element. Real time fluorometric polymerase chain reaction allows amplification of only the DNA fragments that have been successfully ligated together.

One such example is demonstrated in the United States patent application number 20020064779. In the '779 application, a sensitive, rapid and convenient assays for detection and/or quantification of one or several analyte(s) in solution using so called proximity probes is described. The proximity probes contain a binding moiety and a nucleic acid. The nucleic acid from one proximity probe is only capable of interaction with the nucleic acid from the other proximity probe when these are in close proximity, i.e. have bound to the analytes for which they are specific. The '779 application relates to methods and kits for proximity probing and are performed in solution without the need of a solid phase.

Another example can be found in the United States patent application number 20050003361. Here, the application relates to sensitive, rapid and convenient assays for detection and or quantification of one or more analyte(s) in solution using multivalent proximity probes. The proximity probes each comprise several binding moieties, such as antibodies, and associated nucleic acid(s). When the binding moieties have bound to their analyte(s), the nucleic acids on opposite proximity probes interact with each other and a signal is generated based on this interaction. The multivalent proximity probes are especially valuable for highly sensitive and specific protein detection.

However, the present proximity ligation technology requires the use of DNA probes for ligation via the connector nucleotide due to the fact that T4 DNA ligase is not proficient in ligating RNA sequences together. The present inventors recognize that it would be highly desirable to have a method that is superior to the existing technology.

### SUMMARY OF THE INVENTION

In one embodiment the present invention includes compositions and methods for detecting a target in a sample by binding a first and a second ribonucleic acid probe, each of which binds specifically to the target, wherein the first and second probes each comprise a ribonucleic acid tail; ligating the first and second ribonucleic acids tails thereby producing a ligated ribonucleic acid template; and performing amplification of the ribonucleic acid template across the first and second ribonucleic acids. The one aspect the ligation is via a protein ligase, such as T4 DNA ligase, chemical ligation or a nucleic acid ligase such as a ribozyme or deoxyribozyme. Ligation may be accomplished using a template independent ligase, e.g., a T4 RNA ligase 1 or 2. In one aspect, the ligation is in trans or in cis. The method may also probes with ribonucleic acid tails of the first and the second probes that have a complementarity of x bases, wherein x is 0 to 30. The target may be a protein, antibodies, lectins, cell surface receptors, peptides, carbohydrates, nucleic acids such as aptamers, combinatorially derived protein from phage display or ribosome display, or combinations thereof. In one aspect, the first, the second or both the first and second ribonucleic acids are attached to a protein, antibody, lectin, cell surface receptor, peptide, carbohydrate, nucleic acid, combinatorially derived protein from phage display or ribosome display, or combinations thereof. The method may also include the step of adding a nucleic acid splint between the first and second nucleic acid probes. The nucleic acid splint may also include a first region of complementarity to the nucleic acid tail of the first probe, and a second region of complementarity to the nucleic acid tail of the second probe. In one aspect, the amplification is reverse-transcriptase polymerase chain reaction, e.g., a real-time polymerase chain reaction amplification, amplification that is qualitative, quantitative or both qualitative and quantitative.

The method of the present invention may be used to detect target such as, e.g., eukaryotic cell, a prokaryotic cell, a fungal cell, a cell infected with a pathogen, a pathogen, a diseased cell or a cancer cell. The first and second probes may bind to the target directly, indirectly or covalently. In another aspect, the first probe includes a half hairpin and the second probe includes a sequence that hybridizes to a portion of the half hairpin of the first probe wherein the overlap produces a junction for ligation.

Another embodiment of the present invention includes a method for detecting a target in a sample that includes binding a first ribonucleic acid probe and a second ribonucleic acid probe to the target, wherein the first and second probes each comprise a ribonucleic acid tail; adding a nucleic acid splint that comprises an overlap of one or more complementary basepairs with at least a portion of each of the ribonucleic acid tails of the first and second probes; ligating the first and second ribonucleic acids tails to the nucleic acid splint thereby producing a ligated ribonucleic acid template; performing amplification of the ribonucleic acid template across the first ribonucleic acid, the nucleic acid splint and the second ribonucleic acid to produce an amplification product; and detecting the presence or absence of the amplification product. In one aspect, the ligation is via a protein ligase, such as T4 DNA ligase, chemical ligation or a nucleic acid ligase such as a ribozyme or deoxyribozyme. The ligation may be accomplished with a template independent ligase, e.g., a T4 RNA ligase 1 or 2. Ligation may be in trans or in cis.

The present invention also includes a kit for detecting a target in a sample that includes a first container comprising a first probe that binds specifically to the target, wherein the first probe comprises a ribonucleic acid tail; a second contained comprising a second ribonucleic acid probe that binds specifically to the target, wherein the second probe comprises a ribonucleic acid tail; a third container comprising a ligating reagent; and instructions for using the first and second nucleic acid probes to detect a target. The kit may also include a fourth container a nucleic acid splint that includes one or more basepair complementarity overlap with each of the first and second probes. The kit may provide for ligation using a protein ligase, such as T4 DNA ligase, chemical ligation or a nucleic acid ligase such as a ribozyme or deoxyribozyme.

### BRIEF DESCRIPTION OF THE DRAWINGS

For a more complete understanding of the features and advantages of the present invention, reference is now made to the detailed description of the invention along with the accompanying figures and in which:

FIG. 1 is an illustration of an embodiment of basic components from the present invention.

FIG. 2 is a graph illustrating PCR amplification results from the present invention using T4 RNA Ligase 2.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “target” when used in reference to the polymerase chain reaction, refers to the region of nucleic acid bounded by the primers used for polymerase chain reaction. Thus, the “target” is sought to be sorted oat from other nucleic acid sequences. A “segment” is defined as a region of nucleic acid within the target sequence.

The word “specific” as commonly used in the art has two somewhat different meanings. The practice is followed herein. “Specific” refers generally to the origin of a nucleic acid sequence or to the pattern with which it will hybridize to a genome, e.g., as part of a staining reagent. For example, isolation and cloning of DNA from a specified chromosome results in a “chromosome-specific library”. A peptide and/or aptamer may be “target-specific” in that it binds or interacts with its targets above detectable noise in a sample. Shared sequences are not chromosome-specific to the chromosome from which they were derived in their hybridization properties since they will bind to more than the chromosome of origin. A sequence is “locus specific” if it binds only to the desired portion of a genome. Such sequences include single-copy sequences contained in the target or repetitive sequences, in which the copies are contained predominantly in the selected sequence.

As used herein, the term aptamer refers to nucleic acids having a desirable action on a target. A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. In a preferred embodiment, the action is specific binding affinity for a target molecule, such target molecule being a three dimensional chemical structure other than a polynucleotide that binds to the aptamer through a mechanism which predominantly depends on Watson/Crick base pairing or triple helix binding, wherein the aptamer does not have the known physiological function of being bound by the target molecule.

As used herein, the term “PSMA” refers to prostate specific membrane antigen. The Prostate Specific Membrane Antigen (PSMA) is a 750-amino acid type II transmembrane protein. PSMA is expressed by prostatic epithelial cells and extraprostatic expression has been detected in the brain, kidney, salivary gland and duodenum. (See e.g. Renneberg et al. (1999) Urol. Res. 27(1):23-7; Troyer et al. (1995) Int. J. Cancer 62(5):552-8; Israel et al. (1994) Cancer Res. 54(7):1807-11; Israel et al. (1993) Cancer Res. 53(2):227-30). PSMA is a carboxypeptidase which cleaves N-acetyl-asp-glu. PSMA has three domains: a 19-amino acid cytoplasmic domain, a 24-amino acid transmembrane domain, and a 707-amino acid extracellular domain. A monoclonal antibody specific to the cytoplasmic domain, 7E11.C5, has been adapted for in vivo imaging of prostatic cancer through radiolabeling with indium-111. (Elgamal et al. (1998) Prostate 37(4):261-9; Lamb and Faulds (1998) Drugs Aging 12(4):293-304). The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

Polymerase Chain Reaction (PCR) and Real-Time PCR. U.S. Pat. Nos. 4,683,202, 4,683,195, 4,800,159, and 4,965,188, relevant portions incorporated herein by reference disclose conventional PCR techniques. PCR typically employs at least one oligonucleotide primer that binds to a selected nucleic acid template (e.g., DNA or RNA). Primers useful in the present invention include oligonucleotide primers capable of acting as a point of initiation of nucleic acid synthesis within or adjacent to oligonucleotide sequences. A primer can be made from a variety of conventional methods, e.g., synthetically. Primers are typically single-stranded for maximum efficiency in amplification, but a primer can be double-stranded. Double-stranded primers are first denatured (e.g., treated with heat) to separate the strands before use in amplification. Primers can be designed to amplify a nucleotide sequence from a particular species of microbe such as, e.g., B. anthracis, or can be designed to amplify a sequence from more than one species of microbe. Primers that can be used to amplify a nucleotide

The proximity ligation assay, PLA, is a novel variant of immunoPCR in which only adjacent binding events are amplified. In the assay, two affinity probes bearing oligonucleotide tails bound to adjacent sites on a protein or cell surface are ligated together to form a unique amplicon detectable by PCR. Because the assay relies on two specific binding events, the level of background (target independent) ligation is exceeding low. Further, the requirement for two oligonucleotides to be brought together by a bridge reduces background dramatically and is a primary driver for increased sensitivity.

PLA has previously been shown capable of detecting zeptomole amounts of proteins^{1,2}, as few as one bacterial spore³, and exceedingly low levels (˜5 infectious particles) of both viral and bacterial pathogens⁴. In a number of these applications DNA aptamers had been used as PLA probes but RNA aptamers had not, in part because RNA is a poor substrate for T4 DNA ligase. As such, the system is limited to probes constructed of DNA. The present inventors recognize there is a need for a novel method which allows for the use of ribonucleic acid probes capable of binding to a target. The probes contain ribonucleic acid tails which are ligated by an enzyme. The binding event may be confirmed by using an amplification technique.

Target(s) for the present invention may include, but not limited to protein, antibodies, lectins, cell surface receptors, peptides, carbohydrates, nucleic acids such as aptamers, combinatorially derived protein from phage display or ribosome display, or any combinations thereof. Targets may also be a eukaryotic cell, a prokaryotic cell, a fungal cell, a cell infected with a pathogen, a pathogen, a diseased cell or a cancer cell or portions thereof.

In one embodiment, the present invention may be used as a diagnostic tool using RNA aptamers as ribonucleic acid probes that can specifically bind targets on their surface. If the probes are localized adjacent to one another on the target, they may be ligated to generate an unique amplicon that can be reverse transcribed and detected in real-time PCR. The detection of the reverse transcribed template is a signal for detection of the specific target bound and recognized by the RNA aptamers used.

RNA aptamers can be easily generated against almost any target including complex targets such as cancer cells. These aptamers can used to not only bind their specific target but also as reagents directly without the need of additional DNA probes.

In another embodiment of the present invention, the RNA aptamers may be extended on their 3′ and 5′ ends with suitable RNA extensions and can be directly ligated on proximity to one another; these tails may be optionally brought together by a splint oligonucleotide. The ligation event may utilize a template independent RNA ligase to directly ligate two adjacent ribonucleic acid tails. Reverse transcriptase and PCR may be used to directly amplify the proximity event.

Yet in another embodiment of the present invention, T4 RNA ligase 2 is used as the template dependent enzyme for ligating the ribonucleic tails of the ribonucleic acid probes.⁵ A nucleic acid splint is added before the addition of the T4 RNA ligase 2. The ribonucleic acid tails from the probes are complementary to the nucleic acid splint. Typically, the nucleic acid splint contains a first region of complementarity to the nucleic acid tail of the first probe, and a second region of complementarity to the nucleic acid tail of the second probe. In one example of the present invention, RNA aptamers are used as proximity ligation assay reagents without the need of additional DNA probes for ligation. Target used are cells with prostate specific membrane antigen (PSMA) on their surfaces. The anti-PSMA RNA aptamers are extended on their 3′ and 5′ ends with suitable RNA extensions and are directly ligated to one another with the use of the enzyme T4 RNA ligase 2. Finally, real-time PCR is used as the amplification technique.

Materials and Methods. Cell lines. LNCaP (ATCC CRL-1740) and PC3 (ATCC CRL-1435) cells were obtained from the American Type Culture Collection, and cultured according to ATCC specifications.

RNA synthesis. Extended aptamers were generated by runoff transcription from double stranded DNA templates using the Y639F mutant T7 RNA polymerase⁶. Transcription reactions were carried out in 1× transcription buffer (40 mM Tris, pH 8.0, 12 mM MgCl₂, 5 mM DTT, 1 mM spermidine chloride, 4% (w/v) polyethylene glycol 8000, and 0.002% Triton X-100) and contained 1 mM ATP and GTP, 4 mM 2′ fluoro-CTP (2′ F-CTP) and 2′ Fluoro-UTP (2′ F-UTP). Transcriptions went overnight at 42° C., were DNase I treated at 37° C. for 10 min, and RNAs were purified on denaturing (7M urea) 8% polyacrylamide gels. Following elution and precipitation, RNA concentrations were determined based on absorbance at 260 nm using a Nanodrop ND-1000 spectrophotometer.

Preparation of the 5′PLA probe. 5′ anti-prostate specific membrane antigen (PSMA) PLA probe was deposphorylated using Antarctic phosphatase. The RNA aptamer (18 pmoles) was mixed with 2 μL of enzyme (5000 U/ml) in a final volume of 100 μL of the phosphatase buffer. The dephosphorylation reaction was allowed to proceed at 37° C. for 10 min followed by heat inactivation at 65° C. for 5 min. The dephosphorylated aptamer was purified by phenol chloroform extraction followed by ethanol precipitation. The dephosphorylated aptamer was resuspended in 10 μL of ddH₂O, 2 μL of buffer, 20 pmoles of ATP and 1 μL of T4 polynucleotide kinase in a final volume of 20 μL. The kinase reaction was allowed to proceed at 37° C. for 30 min after which the reaction was stopped by the addition of 1 μL of 500 mM EDTA (pH 8.0). The reaction volume was raised to 30 μL with ddH₂O and purified using a microspin G-25 column. The recovered RNA was quantitated using a nanodrop.

PLA reactions. LNCaP and PC3 cells were grown to ˜80% confluence and then treated with trypsin for 5 min at 37° C. Trypsinized cells were washed once with media plus 10% FBS, pelleted by centrifugation at 1500 rpm for 5 min at 4° C., washed with 1 ml of PBS+0.5 mM CaCl₂ and then resuspended in 1 ml of PBS+0.5 mM CaCl₂. Cells were counted using a haemocytometer, and diluted to a concentration of 1,000 cells per microliter.

PLA reactions were carried out by mixing equal concentrations of the 5′monophosphate and 3′-PLA probes (0.01 nM to 10 nM final concentrations) with 1,000 LNCaP, PC3, or no cells in a final volume of 11 μL of PBS+0.5 mM CaCl₂ and 10 mM MgCl₂. Aptamers and cells were preincubated for 1 hr prior to ligation. Ligation was carried out using 0.02 μL of T4 RNA ligase 2 at a concentration of 10,000 U/μL, 2 μl 10× buffer (500 mM Tris-HCl, pH 7.5 20 mM MgCl₂, 10 mM DTT and 4 mM ATP), 400 μM splint and ddH₂O in a final volume of 20 μL per reaction. The ligation reaction was allowed to proceed at 37° C. for 10 min. Following ligation, 10 μL of the ligation mix was reverse transcribed into DNA using the reverse primer (5′ CGCATCGTCCTCCCATTT 3′) (SEQ ID NO.: 1) in a final volume of 20 μL. 10 μL of the reverse transcribed ligation mix was transferred into 40 μL PCR mix, 4 mM MgCl₂, 500 nM primers, 75 nM TaqMan probe, 0.2 mM dNTPs, and 1.5 U AmpliTaq and amplified on a 7300HT Real-time PCR system using the following cycling parameters: 95° C. for 10 min followed by 50 cycles at 95° C. for 1 min and 60° C. for 1 min. Signals were represented in the form of delta C(T); delta C(T)=C(T) no cells−C(T) cells.

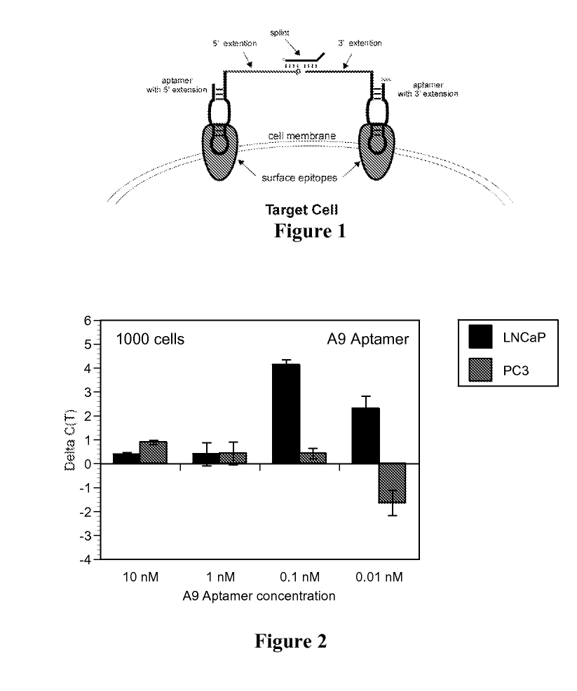

FIG. 1 shows a schematic diagram of the components of the current invention. An anti-PSMA aptamer 10 is extended at the 5′ end with a 5′ ribonucleic acid tail 12; another anti-PSMA aptamer 14 is extended at the 3′ end with a 3′ ribonucleic acid tail 16. The 5′aptamer bears a monophosphate (P) 18. When bound to adjacent sites on a cell surface, ligation and subsequent amplification are mediated by the addition of a splint oligonucleotide 20 and T4 RNA Ligase 2. The 5′ extended aptamer PLA probes was dephophorylated to remove the 5′ triphosphate and subsequently kinased to add a single phosphate group to the 5′ end of the RNA in order to allow for efficient ligation.

In FIG. 2, a strong LNCaP specific PCR amplification signal was observed for reactions carried out at both 0.01 and 0.1 nM aptamer PLA probes. Reactions were carried out with 1,000 LNCaP, PC3 or no cells. The splint oligonucleotide concentration was maintained at 400 pM. Delta C(T) represents the difference between the C(T) value of the background amplification reaction (no cells) and amplification in the presence of cells. Importantly, when reactions were conducted in the absence of the splint oligonucleotide or in the absence of T4 RNA ligase 2 no cell specific signal were detected indicating that the signal is ligation specific.

The present invention demonstrates the direct ligation of two RNA sequences that allows RNA aptamers or other affinity reagents bearing RNA or modified RNA tails to be easily adapted to PLA without the need for the design of DNA probes. In the case of RNA aptamers, PLA probes can now be directly produced using in vitro selection. In addition, RNA aptamers selected against a variety of targets can be used simultaneously for detection of individual targets from a mixture by multiplexed PLA.

The present invention resolves the issue of ligating RNA sequences together with a connector nucleotide without having to rely on additional DNA probes for amplicon formation. By solving this problem, target binding can now be directly coupled with target detection via PCR amplification.

In certain embodiments, the present invention includes a kit for detecting a target in a sample. This kit contains a first container having a first probe that binds specifically to the target, a second container having a second ribonucleic acid probe that binds specifically to the target, a third container having a ligating reagent, and instructions for using the first and second nucleic acid probes to detect the target. The first and second probe each has a ribonucleic acid tail. The ligation may be in cis or in trans, and the ligating reagent may include, but not limited to, a protein ligase such as T4 DNA ligase, a nucleic acid ligase such as a ribozyme or deoxyribozyme, a template independent ligase such as T4 RNA ligase 1 or 2, and reagents that induce chemical ligation or combinations thereof.

Optionally, the kit may include a forth container with a nucleic acid splint inside that contains one or more basepair complementarity overlap with each of the first and second probes or their ribonucleic tails.

The ribonucleic acid tails of the first and second probes in the kit may have different length of complementarity. In one embodiment, the ribonucleic acid tails of the first and the second probes have a complementarity between 0 to 30 bases.

In certain embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Fredriksson S, Gullberg M, Jarvius J, Olsson C, Pietras K, Gustafsdottir S M, Ostman A, Landegren U (2002) Protein detection using proximity-dependent DNA ligation assays. Nat Biotechnol 20:473-7
2. Gullberg M, Gustafsdottir S M, Schallmeiner E, Jarvius J, Bjarnegard M, Betsholtz C, Landegren U, Fredriksson S (2004) Cytokine detection by antibody-based proximity ligation. Proc Natl Acad Sci USA 101:8420-4
3. Pai S, Ellington A D, Levy M (2005) Proximity ligation assays with peptide conjugate ‘burrs’ for the sensitive detection of spores. Nucleic Acids Res 33:e162
4. Gustafsdottir S M, Nordengrahn A, Fredriksson S, Wallgren P, Rivera E, Schallmeiner E, Merza M, Landegren U (2006) Detection of individual microbial pathogens by proximity ligation. Clin Chem 52:1152-60
5. Nandakumar J, Ho C K, Lima C D, Shuman S (2004) RNA substrate specificity and structure-guided mutational analysis of bacteriophage T4 RNA ligase 2. J Biol Chem 279:31337-47
6. Padilla R, Sousa R (1999) Efficient synthesis of nucleic acids heavily modified with non-canonical ribose 2′-groups using a mutantT7 RNA polymerase (RNAP). Nucleic Acids Res 27:1561-3

What is claimed is:

1. A method for detecting a target in a sample comprising:
binding a first and a second ribonucleic acid probe, each of which binds specifically to the target, wherein the first and second probes each comprise a ribonucleic acid tail;
ligating the first and second ribonucleic acids tails thereby producing a ligated ribonucleic acid template; and
performing amplification of the ribonucleic acid template across the first and second ribonucleic acids.

2. The method of claim 1, wherein the ligation is via a protein ligase, such as T4 DNA ligase, chemical ligation or a nucleic acid ligase such as a ribozyme or deoxyribozyme.

3. The method of claim 1, wherein the ligation is with a template independent ligase.

4. The method of claim 3, wherein the template independent ligase is a T4 RNA ligase 1 or 2.

5. The method of claim 1, wherein the ligation is in trans or in cis.

6. The method of claim 1, wherein the ribonucleic acid tails of the first and the second probes have a complementarity of x bases, wherein x is 0 to 30.

7. The method of claim 1, wherein the target comprises a protein, antibody, lectin, cell surface receptor, peptide, carbohydrate, nucleic acid, combinatorially derived protein from phage display or ribosome display, or combinations thereof.

8. The method of claim 1, wherein the first, the second or both the first and second ribonucleic acids are attached to a proteins, antibodies, lectins, cell surface receptors, peptides, carbohydrates, nucleic acids, combinatorially derived protein from phage display or ribosome display, or combinations thereof.

9. The method of claim 1, further comprising the step of adding a nucleic acid splint between the first and second nucleic acid probes.

10. The method of claim 9, wherein the nucleic acid splint comprises a first region of complementarity to the nucleic acid tail of the first probe, and a second region of complementarity to the nucleic acid tail of the second probe.

11. The method of claim 1, wherein the amplification is reverse-transcriptase polymerase chain reaction.

12. The method of claim 11, wherein the polymerase chain reaction comprises a real-time polymerase chain reaction amplification.

13. The method of claim 1, wherein the amplification is qualitative, quantitative or both qualitative and quantitative.

14. The method of claim 1, wherein the target is a eukaryotic cell, a prokaryotic cell, a fungal cell, a cell infected with a pathogen, a pathogen, a diseased cell or a cancer cell.

15. The method of claim 1, wherein the first and second probes bind to the target directly, indirectly or covalently.

16. The method of claim 1, wherein the first probe comprises a half hairpin and the second probe comprises a sequence that hybridizes to a portion of the half hairpin of the first probe wherein the overlap produces a junction for ligation.

17. A method for detecting a target in a sample comprising:
binding a first ribonucleic acid probe and a second ribonucleic acid probe to the target, wherein the first and second probes each comprise a ribonucleic acid tail;
adding a nucleic acid splint that comprises an overlap of one or more complementary basepairs with at least a portion of each of the ribonucleic acid tails of the first and second probes;
ligating the first and second ribonucleic acids tails to the nucleic acid splint thereby producing a ligated ribonucleic acid template;
performing amplification of the ribonucleic acid template across the first ribonucleic acid, the nucleic acid splint and the second ribonucleic acid to produce an amplification product; and
detecting the presence or absence of the amplification product.

18. The method of claim 17, wherein the ligation is via a protein ligase, such as T4 DNA ligase, chemical ligation or a nucleic acid ligase such as a ribozyme or deoxyribozyme.

19. The method of claim 17, wherein the ligation is with a template independent ligase.

20. The method of claim 19, wherein the template independent ligase is a T4 RNA ligase 1 or 2.

21. The method of claim 17, wherein the ligation is in trans or in cis.

22. The method of claim 17, wherein the ribonucleic acid tails of the first and the second probes have a complementarity of x bases, wherein x is 0 to 30.

23. The method of claim 17, wherein the target comprises a protein, antibodies, lectins, cell surface receptors, peptides, carbohydrates, nucleic acids, combinatorially derived protein from phage display or ribosome display, or combinations thereof.

24. The method of claim 17, wherein the nucleic acid splint comprises a first region of complementarity to the nucleic acid tail of the first probe, and a second region of complementarity to the nucleic acid tail of the second probe.

25. The method of claim 17, wherein the amplification is reverse-transcriptase polymerase chain reaction.

26. The method of claim 25, wherein the polymerase chain reaction comprises a real-time polymerase chain reaction amplification

27. The method of claim 17, wherein the target is a eukaryotic cell, a prokaryotic cell, a fungal cell, a cell infected with a pathogen, a pathogen, a diseased cell or a cancer cell.

28. The method of claim 17, wherein the first and second probes bind to the target analyte directly, indirectly or covalently.

29. The method of claim 17, wherein the first probe comprises a half hairpin and the second probe comprises a sequence that hybridizes to a portion of the half hairpin of the first probe wherein the overlap produces a junction for ligation.

30. A kit for detecting a target in a sample comprising:
a first container comprising a first probe that binds specifically to the target, wherein the first probe comprises a ribonucleic acid tail;
a second contained comprising a second ribonucleic acid probe that binds specifically to the target, wherein the second probe comprises a ribonucleic acid tail;
a third container comprising a ligating reagent; and
instructions for using the first and second nucleic acid probes to detect a target.

31. The kit of claim 30, further comprising a fourth container a nucleic acid splint the comprises one or more basepair complementarity overlap with each of the first and second probes.

32. The kit of claim 28, wherein the ligation is via a protein ligase, such as T4 DNA ligase, chemical ligation or a nucleic acid ligase such as a ribozyme or deoxyribozyme.

33. The kit of claim 28, wherein the ligation is with a template independent ligase.

34. The kit of claim 28, wherein the template independent ligase is a T4 RNA ligase 1 or 2.

35. The kit of claim 28, wherein the ligation is in trans or in cis.

36. The kit of claim 28, wherein the ribonucleic acid tails of the first and the second probes have a complementarity of x bases, wherein x is 0 to 30.
